(19) United States
(12) Patent Application Publication
(10) Pub. No.: US 2026/0199579 A1
(43) Pub. Date: Jul. 16, 2026

(54) DEVICE FOR WEIGHING A QUANTITY OF FLUID IN AN EXTRACORPOREAL BLOOD TREATMENT MACHINE

(71) Applicant: B. Braun Avitum AG, Melsungen (DE)

(72) Inventor: Joern Ahrens, Baunatal (DE)

(21) Appl. No.: 19/448,158

(22) Filed: Jan. 14, 2026

(30) Foreign Application Priority Data
Jan. 15, 2025 (DE) 10 2025 101 279.2

(51) Int. Cl. A61M 1/36; G16H 40/63

(52) U.S. Cl. CPC A61M 1/367; G16H 40/63; A61M 2205/3393; A61M 2205/702

(57) ABSTRACT

A device for weighing fluid in a blood treatment machine includes a fastening unit, weighing sensor unit and control unit. The fastening unit mounts a fluid container on a frame. The weighing sensor unit captures a first weight measurement and captures a second weight measurement. The control unit determines a weight for the quantity of fluid in the container based on the first and second weight measurements. The weighing sensor unit captures a first initialization weight measurement and a second initialization weight measurement in an unloaded state of the weighing sensor unit. The control unit compares the first initialization weight measurement with a first limit and compares the second initialization weight measurement with a second limit. The control unit outputs an error message if the first initialization weight measurement is greater than the first limit value and/or the second initialization weight measurement is greater than the second limit value.

### CROSS-REFERENCE TO RELATED APPLICATION

This application claims priority under 35 U.S.C. § 119 to German Application No. 10 2025 101 279.2, filed on January 15, 2025, the content of which is incorporated by reference herein in its entirety

### FIELD

The present disclosure relates to a device for weighing a quantity of fluid in an extracorporeal blood treatment machine, in particular a dialysis machine, a computer-implemented method for operating a device for weighing a fluid, a computer program product, a computer-readable storage medium, and a dialysis machine with such a device.

### BACKGROUND

Load cells for dialysis machines are well known in the prior art and are used in dialysis machines to measure a quantity of fluid. It is important to maintain a balance between the quantity of fluid removed from and supplied to the body. If this is not done with sufficient accuracy, complications may arise for the patient. However, load cells are subject to wear and tear and their properties change over time. For this reason, load cells must be replaced and/or recalibrated from time to time. Due to regulations, this process cannot usually be carried out by medical staff. Instead, specially trained technicians are required. This entails high costs, reduced availability, and, in some cases, less accurate measurement results for the quantity of fluid during operation, thus posing a health risk to patients.

DE 102020210605 A1 discloses a method for correcting an offset. DE 10 2013 016 204 A1 discloses a sensor system for detecting phases and/or phase transitions during peritoneal dialysis treatments.

In this context, it has now become apparent that there is a further need to provide a device for weighing a quantity of fluid in a dialysis machine.

### SUMMARY

It is therefore the task of the present disclosure to provide a device for weighing a quantity of fluid in an extracorporeal blood treatment machine, in particular a dialysis machine. In particular, it is the task of the present disclosure to provide an efficient and accurate device for weighing a quantity of fluid in an extracorporeal blood treatment machine.

The task of the present disclosure is solved by a device for weighing a quantity of fluid in an extracorporeal blood treatment machine. Advantageous embodiments are explained below.

A first aspect of the present disclosure relates to a device for weighing a quantity of fluid in an extracorporeal blood treatment machine, comprising: a fastening unit configured to mount a container, for example a bag, for the quantity of fluid on a frame element; a weighing sensor unit with a first measurement channel and a second measurement channel, wherein the weighing sensor unit is configured to capture a first weight measurement value via the first measurement channel and to capture a second weight measurement value via the second measurement channel; a control unit which is configured to determine a weight for the quantity of fluid in the container on the basis of the captured first weight measurement value and the captured second weight measurement value; wherein the weighing sensor unit is configured to capture a first initialization weight measurement value via the first measurement channel and a second initialization weight measurement value via the second measurement channel in an unloaded state of the weighing sensor unit before weighing the quantity of fluid (S1); wherein the control unit is configured to compare the captured first initialization weight measurement value with a first limit value and to compare the second initialization weight measurement value with a second limit value (S2); wherein the control unit is configured: if an amount of the first initialization weight measurement value is less than an amount of the first limit value and an amount of the second initialization weight measurement value is less than an amount of the second limit value, to set the captured first initialization weight measurement value as an offset for the first measurement channel and to set the captured second initialization weight measurement value as an offset for the second measurement channel (S3); and, wherein the control unit is configured, if an amount of the first initialization weight measurement value is greater than an amount of the first limit value and/or an amount of the second initialization weight measurement value is greater than an amount of the second limit value, to output an error message (S4).

The term “extracorporeal blood treatment machine” refers in particular to a device for performing extracorporeal blood treatment. A dialysis machine is an example of an extracorporeal blood treatment machine. Dialysis comprises cleansing a patient's blood. Purification comprises, among other things, the removal of waste products, excess fluid, and toxins from the blood. In this context, a dialysis machine for hemodialysis is considered in particular. In addition to hemodialysis, the dialysis machine can also be configured for hemofiltration, hemodiafiltration, ultrafiltration, etc. The dialysis machine comprises pumps, sensors, a dialyzer, and containers or bags, such as fluid containers or infusion containers.

The term “fastening unit” refers here to a structural component that is configured to fasten a container, in particular a bag. The fastening unit can be a cantilever arm to which a hook for holding a container is attached. The fastening unit can have an interface for attaching a container. The interface can comprise a hook. The fastening unit can be one-piece or multi-piece.

In this context, the term “container” refers in particular to a hollow body into which a fluid can flow in and/or out. The container may have a rigid housing wall or a flexible housing wall. The container may comprise a bag or be designed as a bag. The container may comprise plastic as a material. The container may be a liquid container, such as an infusion container or a dialysate container.

The term “frame element” refers to a structural component that is configured to arrange or accommodate a fastening unit. The frame element may, for example, be a stand. The frame element may be part of the extracorporeal blood treatment machine, in particular the dialysis machine or dialysis system.

The term “weighing sensor unit” refers here to a unit with at least two measurement channels for capturing a weight, wherein a weight according to the present disclosure is understood to mean a mass. The measurement channel comprises a sensor that is configured to measure a weight. The sensor may comprise one or more of the following: strain gauge sensor, capacitive sensor, piezoelectric sensor. The measurement channel also comprises hardware and software for processing and transmitting the captured measured value. The first and second measurement channels are independent of each other. The first and second measurement channels may be identical or different in design. Preferably, the first and second measurement channels are different in design.

In this context, the term “limit value” refers to a predetermined value that must not be exceeded. The limit value can be specified specifically for the respective first or second measurement channel. The first limit value and the second limit value can be the same or different. Preferably, the first limit value and the second limit value are different. The first limit value and the second limit value can be, for example, + - 20g. The first limit value and the second limit value specify a maximum adjustment value for the offset. For example, the offset may not be changed by 24g in one direction during a new adjustment. For example, the offset may be changed by 17g during a new adjustment.

The term “offset” refers to a measured value of a measurement channel of the weighing sensor unit in an unloaded state.

The control unit can, for example, determine a weight for the quantity of fluid in the container by using the first weight measurement value as a value for the weight for the quantity of fluid in the container. The control unit can, for example, determine a weight for the quantity of fluid in the container by using an average value from the first weight measurement value and the second weight measurement value as the value for the weight of the quantity of fluid in the container. The control unit may further be configured to form a difference between the first weight measurement value and the second weight measurement value and to compare this with a working limit value. The working limit value may, for example, be 10g. The control unit may further be configured to output an error message (e.g., excessive measurement deviation between the measurement channels) if the difference exceeds the working limit value. The control unit may further be configured to output the weight for the quantity of fluid in the container if the difference does not exceed the working limit value.

The disclosure is based on the knowledge that a load cell naturally exhibits an offset when weighing a quantity of fluid. This means that even when unloaded, a measurement channel of the load cell displays a measured value. This offset can change due to wear and tear, changes in environmental conditions, incorrect setup of the device for weighing the quantity of fluid (e.g., incorrect hook or additional load), among other things. This offset is usually compared with a limit value, and if the offset is below the limit value, measurement is performed without calibration using the old, originally specified offset. The old, originally specified offset comes from a calibration at the manufacturing plant or from commissioning by a certified measurement technician. If the limit value is exceeded, an error message is output. However, this has the disadvantage that, on the one hand, measurements are inaccurate and, on the other hand, gradual changes in environmental conditions are overrated. The present disclosure now proposes to record an initialization weight measurement value in an unloaded state before each measurement for the first and second measurement channels, compare this with a limit value, and, if this initialization weight measurement value is below the limit value, use it as the new offset. This corresponds to a new calibration, but one that does not require a measurement technician. If one or both measurement channels are exceeded, an error message is output. In this way, measurement accuracy can be increased, as a more accurate offset is used in each case, and the availability of the load cell can be increased, as an error message in the event of gradual deterioration is avoided by means of a new calibration. Furthermore, the calibration described above makes it possible to set the first and/or second limit value higher before each measurement run. This can also have a positive effect on availability.

According to a preferred embodiment, the error message may include a request to check the device with regard to measurement conditions.

The error message may, for example, be displayed on a display of the device. The measurement conditions may comprise the measurement setup and/or the environmental conditions (e.g., temperature, room humidity, solar radiation). The error message may, for example, include notes and/or questions relating to the measurement setup. Questions may comprise, for example, whether there is really no weight hanging on the device or whether the device or the hook of the device is hanging freely. The user can then check the measurement conditions accordingly and decide whether the measurement should be repeated or whether a technician should be called in.

In this way, the availability of the device can be advantageously increased.

According to a preferred embodiment, after checking the device with regard to the measurement conditions, the device may be configured to perform steps S1 to S4 again.

For example, the device can be further configured to receive confirmation of the check by an employee after the check has been performed. This can be done via an interface, such as a control panel, a button, or a human-machine interface (HMI). The device can repeat steps S1 to S4. If the result is positive, the measurement can then be performed successfully. If the result is negative, a new error message with the same content or, if necessary, a note to call in a technician can be displayed. The latter then leads to an inevitable temporary failure of the device.

In this way, the overall availability of the device can be increased, as the necessary inspection by a technician with new, time-consuming calibration can be avoided.

According to a preferred embodiment, the control unit may further be configured to compare a first difference between the first captured initialization weight measurement value and a first initial initialization weight measurement value with a first absolute limit value, to compare a second difference between the second captured initialization weight measurement value and a second initial initialization weight measurement value with a second absolute limit value, if the first difference is less than the first absolute limit value and the second difference is less than the second absolute limit value, to set the captured first initialization weight measurement value as an offset for the first measurement channel and to set the captured second initialization weight measurement value as an offset for the second measurement channel, and if the first difference is greater than the first absolute limit value and/or the second difference is greater than the second absolute limit value, to output an error message.

In this context, the term “initial initialization weight measurement value” refers in particular to a measurement value that was determined during calibration with measuring weights, for example during the manufacture of the weighing sensor unit or the device. The first initial initialization weight measurement value can preferably refer to the first measurement channel. The second initial initialization weight measurement value may preferably relate to the second measurement channel.

The term “absolute limit value” refers in this context to a limit value for a deviation from an initial initialization weight measurement value. The first absolute limit value is, for example, +-100g. The second absolute limit value is, for example, +-100g. The two absolute limit values may be the same or different.

The difference between the first initial initialization weight measurement value and the captured first initialization weight measurement value can advantageously provide information about how far the currently captured initialization weight measurement value deviates from the original first initial initialization weight measurement value. If the first absolute limit value is exceeded, it can be assumed that there is a defect in the measurement channel and/or that the measurement conditions are not suitable. The same applies analogously to the second captured initialization weight measurement value and the second initial initialization weight measurement value.

In this way, a trend can be taken into account in a synergistic and advantageous manner, while at the same time taking into account any excessive changes in the measurement channels compared to the delivery state. This can increase the availability and, at the same time, the reliability of the device.

According to a preferred embodiment, the control unit may further be configured such that, if the first initialization weight measurement value is greater than the first limit value and the second initialization weight measurement value is greater than the second limit value, and if a difference between the first initialization weight measurement value and the second initialization weight measurement value is less than a tolerance limit value, to output an error message with a request to check the measurement conditions.

If both initialization weight measurement values change to the same extent, it can be assumed that the cause lies in the measurement conditions and not in the hardware and/or software of the measurement channels. For example, there may have been an increase in temperature or mechanical stress on the device. The device addresses this situation by performing an additional check of the initialization weight measurement values against each other and then issuing a corresponding error message.

In this way, an error in the measurement conditions can be detected efficiently.

According to a preferred embodiment, the control unit may further be configured to compare, during operation, a difference between the weight determined with the first measurement channel and the weight determined with the second measurement channel with an operating limit value, and to output an error message if the difference is greater than the operating limit value.

The first measurement channel provides, for example, the result for the weight of the quantity of fluid. The second measurement channel can preferably serve to check the first measurement channel. The operating limit value is, for example, +- 10g. In the present case, a comparison of both measurement channels during actual operation additionally checks whether the amplification factor and the associated measured value are correct for weight determination. If the difference between the two weights is too large, this may be an indication that the measurement channel is defective.

In this way, the reliability of the device can be advantageously increased.

According to a preferred embodiment, the control unit may be configured, over the course of the service life of the device, to evaluate a further captured first initialization weight measurement value of the first measurement channel with previously captured first initialization weight measurement values of the first measurement channel with regard to a trend and to evaluate a further captured second initialization weight measurement value of the second measurement channel with previously captured second initialization weight measurement values of the second measurement channel with regard to a trend, wherein the control device is configured in particular to output an error message when a trend has been identified in the first measurement channel and/or second measurement channel.

By analyzing the initialization weight measurement values with regard to a trend, gradually changing conditions can be identified and an error message can be issued at an early stage. The trends may indicate an imminent failure of the device. In this way, the availability of the device can be advantageously increased.

Another aspect of the present disclosure relates to a computer-implemented method for operating a device for weighing a fluid, wherein the device comprises a control unit, a weighing sensor unit with a first measurement channel and a second measurement channel, and a fastening unit for fastening a container, wherein the computer-implemented method comprises the steps of: capturing, before weighing the quantity of fluid in an unloaded state of the weighing sensor unit, an initial initialization weight measurement value via the first measurement channel and a second initialization weight measurement value via the second measurement channel (S1); comparing the captured first initialization weight measurement value with a first limit value and the second initialization weight measurement value with a second limit value (S2); setting the captured first initialization weight measurement value as an offset for the first measurement channel and the captured second initialization weight measurement value as an offset for the second measurement channel if the first initialization weight measurement value is less than the first limit value and the second initialization weight measurement value is less than the second limit value (S3); outputting an error message if the first initialization weight measurement value is greater than the first limit value and/or the second initialization weight measurement value is greater than the second limit value (S4).

This increases measurement accuracy, as a more precise offset is used in each case, and also increases the availability of the device for weighing fluids, as error messages caused by gradual deterioration are avoided by recalibration. Furthermore, the calibration described above makes it possible to set the first and/or second limit value higher before each measurement run. This can also have a positive effect on availability of the device.

According to a preferred embodiment, the error message may include a request to check the device with regard to measurement conditions.

In this way, the availability of the device can be advantageously increased.

According to a preferred embodiment, after checking the device with regard to the measurement conditions, steps S1 to S4 can be performed again.

In this way, the overall availability of the device can be increased, as the necessary inspection by a technician with new, time-consuming calibration can be avoided.

According to a preferred embodiment, a method may be provided, further comprising: comparing a first difference between the first initialization weight measurement value captured and a first initial initialization weight measurement value with a first absolute limit value; comparing a second difference between the second initialization weight measurement value captured and a second initial initialization weight measurement value with a second absolute limit value; setting the captured initial initialization weight measurement value as an offset for the first measurement channel and the captured second initialization weight measurement value as an offset for the second measurement channel if the first difference is less than the first absolute limit value and the second difference is less than the second absolute limit value; and outputting an error message if the first difference is greater than the first absolute limit value and/or the second difference is greater than the second absolute limit value.

In this way, a trend can be taken into account in a synergistic and advantageous manner, while at the same time taking into account any excessive changes in the measurement channels compared to the delivery state. This can increase the availability and, at the same time, the reliability of the device.

Another aspect of the present disclosure relates to a computer program product comprising instructions that, when executed by a computer or microprocessor, cause the computer or microprocessor to perform the method described in more detail above.

Another aspect relates to a computer-readable storage medium comprising instructions which, when executed by a computer or microprocessor, cause it to perform the method described in more detail above.

A further aspect relates to an extracorporeal blood treatment machine, in particular a dialysis machine, with a device described in more detail above.

The units according to one or more exemplary embodiments may be implemented using hardware, software, and/or a combination thereof. The units can be single-piece or multi-piece. Hardware units can be implemented, for example, by processing circuits such as a processor, a central processing unit (CPU), a controller, an arithmetic logic unit (ALU), a digital signal processor, a microcomputer, a field-programmable gate array (FPGA), a system-on-chip (SoC), a programmable logic device, a microprocessor, or any other device capable of responding to commands and executing them in a specified manner.

The units may comprise one or more interface circuits. In some examples, the interface circuits may include wired or wireless interfaces connected to a local area network (LAN), the Internet, a wide area network (WAN), or combinations thereof. The functionality of a specific unit of the present disclosure may be distributed across multiple units connected via interface circuits.

The units according to one or more exemplary embodiments may also include one or more storage devices. The one or more storage devices may be physical or non-transitory computer-readable storage media, such as random access memory (RAM), read-only memory (ROM), a permanent mass storage device (e.g., a hard disk drive), a solid-state device (e.g., NAND flash), and/or any other data storage mechanism capable of storing and recording data. The one or more storage devices may be set up to store computer programs, program code, instructions, or a combination thereof.

The explanations and advantages of individual embodiments described here also apply mutatis mutandis to the other embodiments. Various exemplary features of the embodiments may be combined according to the present disclosure wherever this is technically useful and feasible.

### BRIEF DESCRIPTION OF THE DRAWINGS

FIG. 1 is a diagram illustrating a device for weighing a fluid in a dialysis machine according to a first embodiment of the present disclosure;

FIG. 2 is a diagram of an offset curve over several measurement cycles without using the device disclosed in the present disclosure;

FIG. 3 is a diagram of the offset curve over several measurement cycles without using a device according to the disclosure;

FIG. 4 is a diagram of a further offset curve over several measurement cycles using a device according to the disclosure; and

FIG. 5 is a diagram comparing a first initialization weight measurement value and a second initialization weight measurement value.

### DETAILED DESCRIPTION

The following describes exemplary embodiments of the present disclosure on the basis of the associated figures.

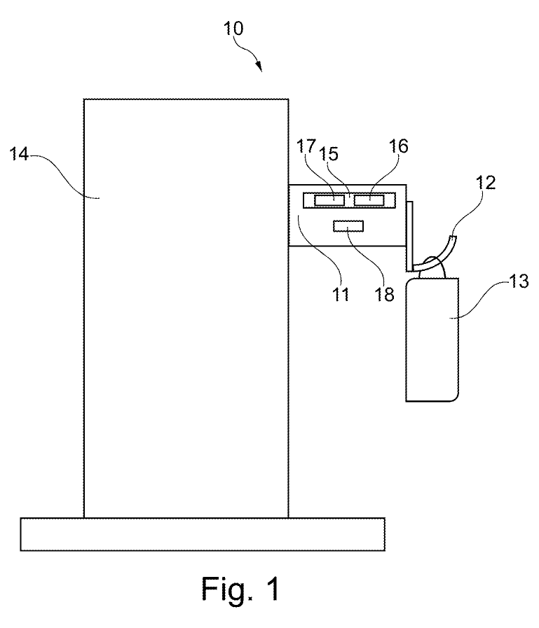

FIG. 1 shows a device 10 for weighing a quantity of fluid in a dialysis machine 14. The quantity of fluid is located in a container 13. From this container 13, the quantity of fluid flows, for example, into a patient (not shown) or into a filter unit (not shown) of the dialysis machine 14. Alternatively, a quantity of fluid, for example used dialysis fluid/dialysate, can also flow into this container 13. For this purpose, the container 13 has at least one opening (inlet opening or outlet opening). The device 10 has a fastening unit 11 for holding the container 13. The fastening unit 11 comprises a hook 12 as an interface for holding the container 13. This hook can be connected to the fastening unit 11, for example, by a positive-locking, a force fit, or a material fit. The fastening unit 11 is connected to a frame element 14. The frame element 14 corresponds to the dialysis machine 14. The device 10 also comprises a weighing sensor unit 15 with two measurement channels 16 and 17 for measuring a first weight measurement value and for measuring a second weight measurement value. The two measurement channels measure the load acting on the fastening unit 11 and/or the hook 12. In the present case, the two measurement channels each have strain gauge sensors, hardware, and software for determining the weight measurement values. The measurement channels are identical in the present case. Alternatively, they may also differ. The device 10 also has a control unit 18. The control unit 18 is located in the fastening unit 11. Alternatively, it may also be arranged separately from the latter, for example in the dialysis machine 14. The control unit 18 is configured to determine a weight for the quantity of fluid in the container 13 on the basis of the captured first weight measurement value and the captured second weight measurement value. In the present case, the control unit 18 determines a weight for the quantity of fluid in the container 13 by using the first weight measurement value as the value for the weight of the quantity of fluid in the container 13. The control unit 18 is further configured to form a difference between the first weight measurement value and the second weight measurement value and to compare this with a working limit value. The working limit value is 10g. The control unit 18 is further configured to output an error message (e.g., excessive measurement deviation between the measurement channels) if the difference exceeds the working limit value. The control unit 18 is further configured to output the weight for the quantity of fluid in the container 13 if the difference does not exceed the working limit value. The weighing sensor unit 15 is configured to capture a first initialization weight measurement value via the first measurement channel 16 and a second initialization weight measurement value via the second measurement channel 17 in an unloaded state of the weighing sensor unit 15 before weighing the quantity of fluid (S1); In the present case, the weighing sensor unit 15 is unloaded when no container 13 is hanging on the hook 12. The control unit 18 is further configured to compare the captured first initialization weight measurement value with a first limit value and to compare the second initialization weight measurement value with a second limit value (S2). In the present case, the first limit value and the second limit value are preferably +- 20g each. Furthermore, the control unit 18 is configured if the first initialization weight measurement value is less than the first limit value and the second initialization weight measurement value is less than the second limit value, to set the captured first initialization weight measurement value as the offset for the first measurement channel and to set the captured second initialization weight measurement value as the offset for the second measurement channel (S3). In the present case, the first initialization weight measurement value captured is, for example, 17g. Thus, the first initialization weight measurement value is less than the first limit value, which is +- 20g. In this case, the second initialization weight measurement value captured is, for example, 23g. Thus, the second initialization weight measurement value is greater than the second limit value, which is +- 20g. Thus, in this case, the control unit 18 does not set any new offsets for the first measurement channel and the second measurement channel. Furthermore, the control unit 18 is configured to output an error message (S4) if the first initialization weight measurement value is greater than the first limit value and/or the second initialization weight measurement value is greater than the second limit value. In this case, the second initialization weight measurement value of 23g is greater than the second limit value of +- 20g. The control unit 18 therefore outputs an error message. In this case, the error message comprises a request to check the device with regard to measurement conditions. In this case, the error message is displayed on a display of the device 10. The error message may optionally also include, for example, a check of the hook 12 or the device 10 as a whole. Alternatively, if both initialization weight measurement values were less than the corresponding limit values, the control unit 18 would use the two initialization weight measurement values as offsets for the respective measurement channels, so that both measurement channels would display a weight of 0g in the unloaded state.

Furthermore, the control unit 18 compares a first difference between the first captured initialization weight measurement value and a first initial initialization weight measurement value with a first absolute limit value. The first initial initialization weight measurement value was determined during the manufacture of the device. The first absolute limit value is +-100g in the present case. Furthermore, the control unit 18 compares a second difference between the second captured initialization weight measurement value and a second initial initialization weight measurement value with a second absolute limit value. The second initial initialization measurement value was determined during the manufacture of the device. The second absolute limit value is +-100g in the present case. In this case, the differences are smaller than the absolute limit values so that no error message is issued.

Furthermore, if the first initialization weight measurement value is greater than the first limit value and the second initialization weight measurement value is greater than the second limit value, the control unit 18 determines a difference between the first initialization weight measurement value and the second initialization weight measurement value and compares this difference with a tolerance limit value. The tolerance limit value is, for example, 5g. If the difference is less than the tolerance limit value, the control unit 18 issues an error message with a request to check the measurement conditions. In this case, only the second initialization weight measurement value is greater than the second limit value, so this check is not performed.

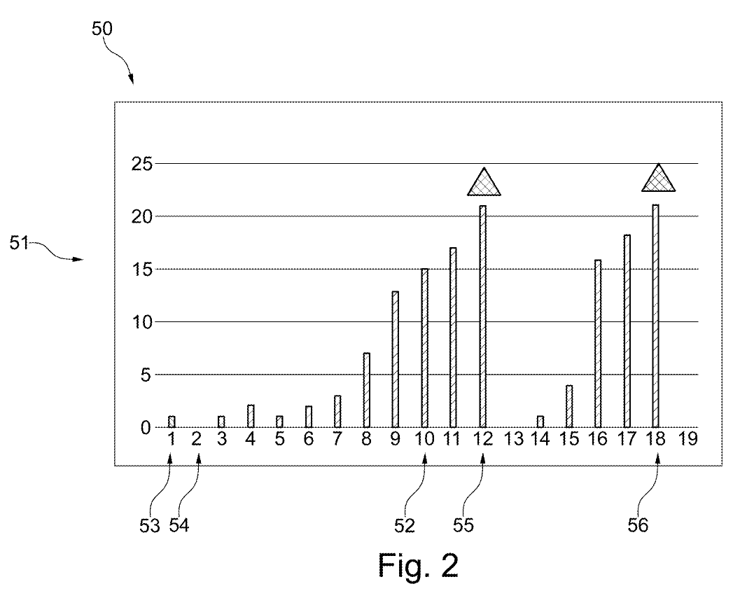

FIG. 2 shows the offset curve 50 over a plurality of measurements for a device for weighing a quantity of fluid that does not correspond to the device disclosed. The offset for an unloaded state of the device is plotted on the vertical axis 51. The number of measurements with the device is plotted on the horizontal axis 52. During the first measurement 53, for example in the production plant, a corresponding offset is determined and readjusted so that the device displays zero as the measured value during the second measurement 54. The offset increases over the course of the measurements. During the twelfth measurement 55, the measured value in the unloaded state exceeds a limit value. The limit value is, for example, 20g in this case. In this case, a technician must then calibrate the device (e.g., with a measuring weight) so that it again displays the value zero as the measured value during the thirteenth measurement. After that, the offset increases again over the individual measurements until the limit value is exceeded again during the eighteenth measurement 56.

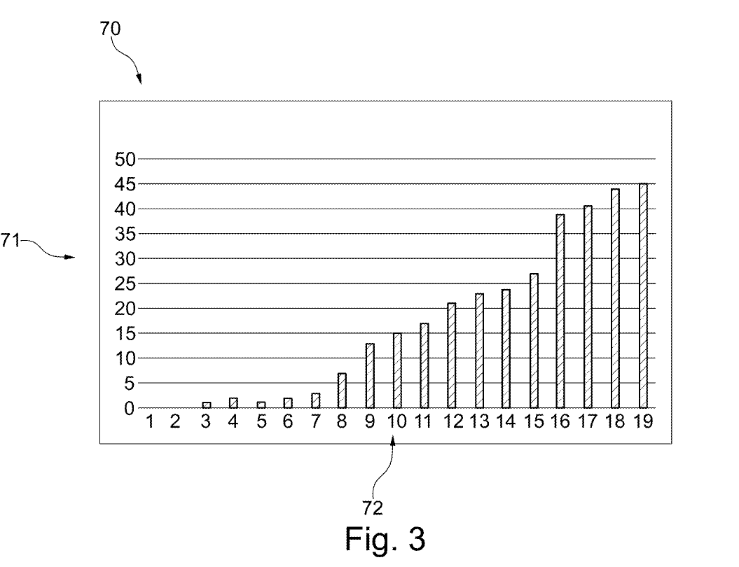

FIG. 3 shows the offset curve 70 over a plurality of measurements for a further device for weighing a quantity of fluid that does not correspond to the device disclosed. The offset for an unloaded state of the device is plotted on the vertical axis 71. The number of measurements with the device is plotted on the horizontal axis 72. Unlike in the case of curve 50, no recalibration is performed by a technician when a threshold value is exceeded. The curve 70 increases continuously.

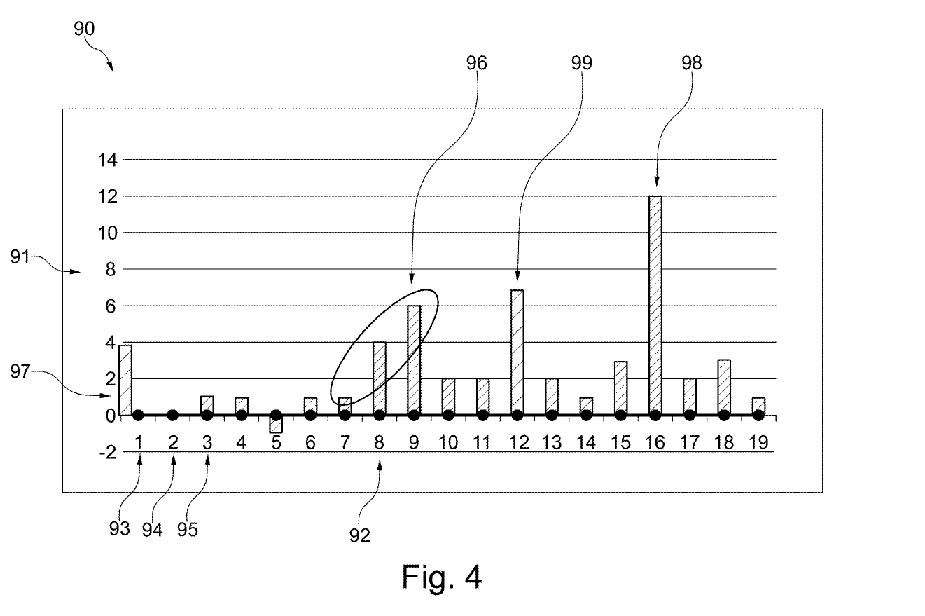

FIG. 4 shows the curve 90 of the offset over a plurality of measurements for a device according to the disclosure for weighing a quantity of fluid. The offset for an unloaded state of the device 10 for the first measurement channel is plotted on the vertical axis 91. At this point, it should be mentioned that the explanations apply analogously to the second measuring channel. The number of measurements with the device 10 is plotted on the horizontal axis 92. This shows, for example, that no new deviation was measured in the first two measurements 93 and 94, but a new deviation was measured in the third measurement 95. This was then chosen as the new offset. Overall, it can be seen that the offset is recalculated before each actual measurement. This can also be referred to as auto-calibration.

Furthermore, it can be seen in region 96 that the measurements are increasing disproportionately. The control unit 18 evaluates the respective initialization weight measurement values with regard to such an increase or trend, for example by forming the differences between successive initialization weight measurement values and comparing them with each other. If such a trend is detected, the control unit 18 issues a corresponding error message. The error message may, for example, include a request to check the environmental conditions.

Furthermore, an initial initialization weight measurement value of 97 is plotted in the diagram. The initial initialization measurement 97 is 4g in this case. For example, the first absolute limit value mentioned above is 7g. The initial initialization weight measurement value of 98 is 12g. A corresponding difference between the first initialization weight measurement value 98 and the first initial initialization weight measurement value is 8g in this case and is therefore greater than the first absolute limit value of 7g. In this case, an error message would be issued, requiring a technician to be called in to recalibrate the device.

Furthermore, the first threshold value in this case is 6g. The measured value 99 would exceed the first threshold value of 6 in this case, resulting in an error message being issued.

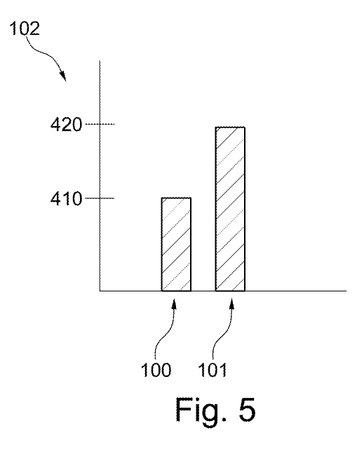

FIG. 5 shows a comparison of a weight 100 of the first measurement channel 16 and a weight 101 of the second measurement channel 17, which were determined during operation of the device 10. The values for the weights are plotted on the vertical axis 102. The difference between the two weights is 10g in this case. The difference is therefore less than the operating limit value of 20g in this case. The control unit 18 therefore does not issue an error message. Otherwise, the control unit 18 would issue an error message.

### List of reference numerals

10 device

11 fastening unit

12 hook

13 container

14 frame element, dialysis machine

15 weighing sensor unit

16 first measurement channel

17 second measurement channel

18 control unit

50, 70, 90 offset curve

51, 71, 91, 102 vertical axis

52, 72, 92 horizontal axis

53, 54, 55, 56 individual measurements without mechanical stress

93, 94, 95 individual measurement with subsequent auto-calibration

96 range with trend

97 initial initialization weight measurement value

98 individual measurement greater than limit

99 measurement

101 weight of first measurement channel

102 weight of second measurement channel

1. A device for weighing a quantity of fluid in an extracorporeal blood treatment machine, the device comprising:
a fastening unit configured to mount a container for the quantity of fluid on a frame element;
a weighing sensor unit with a first measurement channel and a second measurement channel, the weighing sensor unit being configured to capture a first weight measurement value via the first measurement channel and to capture a second weight measurement value via the second measurement channel; and
a control unit configured to determine a weight for the quantity of fluid in the container based on the first weight measurement value and the second weight measurement value,
the weighing sensor unit being further configured to capture a first initialization weight measurement value via the first measurement channel and a second initialization weight measurement value via the second measurement channel in an unloaded state of the weighing sensor unit before weighing the quantity of fluid,
the control unit being further configured to compare the first initialization weight measurement value with a first limit value and to compare the second initialization weight measurement value with a second limit value, and
the control unit being further configured to:
set the first initialization weight measurement value as an offset for the first measurement channel and set the second initialization weight measurement value as an offset for the second measurement channel when an amount of the first initialization weight measurement value is less than an amount of the first limit value, and when an amount of the second initialization weight measurement value is less than an amount of the second limit value, and
output an error message when the amount of the first initialization weight measurement value is greater than the amount of the first limit value and/or when the amount of the second initialization weight measurement value is greater than the amount of the second limit value.

2. The device according to claim 1, wherein the error message comprises a request to check the device with regard to measurement conditions.

3. The device according to claim 1, wherein the control unit is further configured to:
compare a first difference between the first initialization weight measurement value and a first initial initialization weight measurement value with a first absolute limit value,
compare a second difference between the second initialization weight measurement value and a second initial initialization weight measurement value with a second absolute limit value,
set the first initialization weight measurement value as the offset for the first measurement channel and set the second initialization weight measurement value as the offset for the second measurement channel when the first difference is smaller than a first absolute limit value and the second difference is smaller than a second absolute limit value, and
output an error message when the first difference is greater than the first absolute limit value and/or the second difference is greater than the second absolute limit value.

4. The device according to claim 1, wherein the control unit is further configured to output an error message with a request to check measurement conditions when:
the first initialization weight measurement value is greater than the first limit value and the second initialization weight measurement value is greater than the second limit value, and
a difference of the first initialization weight measurement value and the second initialization weight measurement value is smaller than a tolerance limit value.

5. The device according to claim 1, wherein the control unit is further configured to compare, during operation, a difference between the weight determined with the first measurement channel and the weight determined with the second measurement channel with an operating limit value, and to output an error message if the difference is greater than the operating limit value.

6. The device according to claim 1, wherein the control unit is configured to evaluate a further captured first initialization weight measurement value of the first measurement channel with previously captured first initialization weight measurement values of the first measurement channel with regard to a trend and to evaluate a further captured second initialization weight measurement value of the second measurement channel with previously captured second initialization weight measurement values of the second measurement channel with regard to a trend.

7. The device according to claim 6, wherein the control unit is configured to output an error message when a trend has been identified in the first measurement channel and/or second measurement channel.

8. An extracorporeal blood treatment machine comprising the device according to claim 1.

9. A computer-implemented method for operating a device for weighing a fluid, wherein the device comprises a control unit, a weighing sensor unit with a first measurement channel and a second measurement channel, and a fastening unit for attaching a container, the computer-implemented method comprising the steps of:
A) capturing, before weighing a quantity of fluid in an unloaded state of the weighing sensor unit, a first initialization weight measurement value via the first measurement channel and a second initialization weight measurement value via the second measurement channel;
B) comparing the first initialization weight measurement value with a first limit value and the second initialization weight measurement value with a second limit value;
C) setting the first initialization weight measurement value as an offset for the first measurement channel and the second initialization weight measurement value as an offset for the second measurement channel if the first initialization weight measurement value is less than the first limit value and the second initialization weight measurement value is less than the second limit value; and
D) outputting an error message if the first initialization weight measurement value is greater than the first limit value and/or the second initialization weight measurement value is greater than the second limit value.

10. The computer-implemented method according to claim 9, wherein the error message comprises a request to check the device with regard to measurement conditions.

11. The computer-implemented method according to claim 9, wherein, after checking the device with regard to measurement conditions, the device performs steps A), B), C) and D) again.

12. The computer-implemented method according to claim 9, further comprising the steps of:
E) comparing a first difference between the first initialization weight measurement value and a first initial initialization weight measurement value with a first absolute limit value;
F) comparing a second difference between the second initialization weight measurement value and a second initial initialization weight measurement value with a second absolute limit value;
G) setting the first initialization weight measurement value as the offset for the first measurement channel and the second initialization weight measurement value as the offset for the second measurement channel if the first difference is smaller than the first absolute limit value and the second difference is smaller than the second absolute limit value; and
H) outputting an error message if the first difference is greater than the first absolute limit value and/or the second difference is greater than the second absolute limit value.

13. A non-transitory computer-readable storage medium comprising instructions that, when executed by a computer or microprocessor, cause the computer or microprocessor to perform the computer-implemented method according to claim 9.
